(19) United States
(12) Patent Application Publication
(10) Pub. No.: US 2025/0252762 A1
(43) Pub. Date: Aug. 7, 2025

(54) ENHANCING DATA VIEWS FOR REMOTE INTENSIVE CARE UNITS

(71) Applicant: KONINKLIJKE PHILIPS N.V., EINDHOVEN (NL)

(72) Inventors: SHENG JIN, SHANGHAI (CN); CONG TIAN, SHANGHAI (CN)

(21) Appl. No.: 18/856,911

(22) PCT Filed: Apr. 11, 2023
(86) PCT No.: PCT/EP2023/059355

§ 371 Date: Oct. 15, 2024

Related U.S. Application Data
(60) Provisional application No. 63/331,911, filed on Apr. 18, 2022.

(51) Int. Cl. G06V 20/70; G06V 20/52; G16H 40/67; H04N 7/18

(52) U.S. Cl. CPC G06V 20/70; G06V 20/52; G16H 40/67; H04N 7/181; G06V 2201/03

(57) ABSTRACT

A system for enhancing data views for remote intensive care units includes a central computer comprising a memory that stores instructions and a processor that executes the instructions. When executed by the processor, the instructions cause the central computer to: receive, from a first remote intensive care unit, data generated by a first camera at the first remote intensive care unit; determine, from the data generated by the first camera at the first remote intensive care unit, a type of a first medical device within a view of the first camera at the first remote intensive care unit; determine, based on the type of the first medical device, a layout of labels for information displayed by the first medical device; generate labels for information displayed by the first medical device; and supplement video from the first camera with the labels for information displayed by the first medical device.

### BACKGROUND

An intensive care unit (ICU) is a special department of a health care facility that provides intensive care medicine. Typically, patients in an ICU are assigned to beds and a variety of medical equipment including medical monitors is arranged around the beds. In a typical arrangement, medical equipment including medical monitors is placed on one side of a bed in an ICU, so that the patient may move or be moved on and off the bed on the other side.

A tele-ICU system enables remote clinicians to interact with bedside staff to consult on patient care. Tele-ICU programs concentrate clinical resources in remote care centers such as central monitoring facilities and extend the resources to the bedside via technology. For example, a tele-ICU program may leverage audio/visual (A/V) conferencing and real-time data-streams of patient information from multiple interfaces, independent of the location of the remote care center or the health care facility with the actual ICU. One care team of remote clinicians for a tele-ICU system may manage a large number of geographically distributed ICU locations to exchange health information electronically, in real time. A tele-ICU system is a supplement to and not necessarily a replacement for the bedside team, offering support to increasingly scarce clinical resources.

In some ICUs, not all medical devices are connected to the internet. For example, some patient monitors may not be connected to the internet at all, or at least not in advance of a tele-ICU session. While it may be possible to use camera systems installed near the beds to capture and stream the display/user interface of the medical devices, oftentimes the information of the display/user interface cannot be seen due to limitations of resolution, zoom (magnification) power, lighting environment, and more. As one notable example, labels for information on displays/user interfaces are often significantly less visible than the underlying information for which the labels are provided. For instance, the numerical information for an SpO2 reading may be relatively large, whereas the label labelling the numerical information as an SpO2 reading may be too small to see in the video. As a result, the clinicians at the remote tele-ICU may not be able to determine the type of information they are reading on a screen.

### SUMMARY

According to an aspect of the present disclosure, a system for enhancing data views for remote intensive care units includes a central computer comprising a memory that stores instructions and a processor that executes the instructions. When executed by the processor, the instructions cause the central computer to: receive, from a first remote intensive care unit, data generated by a first camera at the first remote intensive care unit; determine, from the data generated by the first camera at the first remote intensive care unit, a type of a first medical device within a view of the first camera at the first remote intensive care unit; determine, based on the type of the first medical device, a layout of labels for information displayed by the first medical device; generate labels for information displayed by the first medical device; and supplement video from the first camera with the labels for the information displayed by the first medical device.

According to another aspect of the present disclosure, a method for enhancing data views for remote intensive care units includes receiving, by a central computer comprising a memory that stores instructions and a processor that executes the instructions, from a first remote intensive care unit, data generated by a first camera at the first remote intensive care unit. The method further includes determining, from the data generated by the first camera at the first remote intensive care unit, a type of a first medical device within a view of the first camera at the first remote intensive care unit. The method also includes determining, based on the type of the first medical device, a layout of labels for information displayed by the first medical device; generating labels for information displayed by the first medical device; and supplementing video from the first camera with the labels for the information displayed by the first medical device.

According to another aspect of the present disclosure, a tangible non-transitory computer readable storage medium stores a computer program. The computer program, when executed by a processor, causes a central computer to: receive, from a first remote intensive care unit, data generated by a first camera at the first remote intensive care unit; determine, from the data generated by the first camera at the first remote intensive care unit, a type of a first medical device within a view of the first camera at the first remote intensive care unit; determine, based on the type of the first medical device, a layout of labels for information displayed by the first medical device; generate labels for information displayed by the first medical device; and supplement video from the first camera with the labels for the information displayed by the first medical device.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 illustrates a system for enhancing data views for remote intensive care units, in accordance with a representative embodiment.

FIG. 2 illustrates a library for enhancing data views for remote intensive care units, in accordance with a representative embodiment.

FIG. 3A illustrates a method for enhancing data views for remote intensive care units, in accordance with a representative embodiment.

FIG. 3B illustrates a method for enhancing data views for remote intensive care units, in accordance with a representative embodiment.

FIG. 4 illustrates a system for enhancing data views for remote intensive care units, in accordance with a representative embodiment.

FIG. 5 illustrates a system for enhancing data views for remote intensive care units, in accordance with a representative embodiment.

FIG. 6 illustrates a computer system, on which a method for enhancing data views for remote intensive care units is implemented, in accordance with another representative embodiment.

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

As described herein, labels captured by cameras may be automatically enhanced so that viewers of the captured imagery may be enabled to better understand what they are viewing. In the context of remote intensive care units, the labels may correspond to information being displayed on medical equipment, so that clinicians at remote intensive care units are enabled to view and understand the labels for the information displayed on the medical equipment.

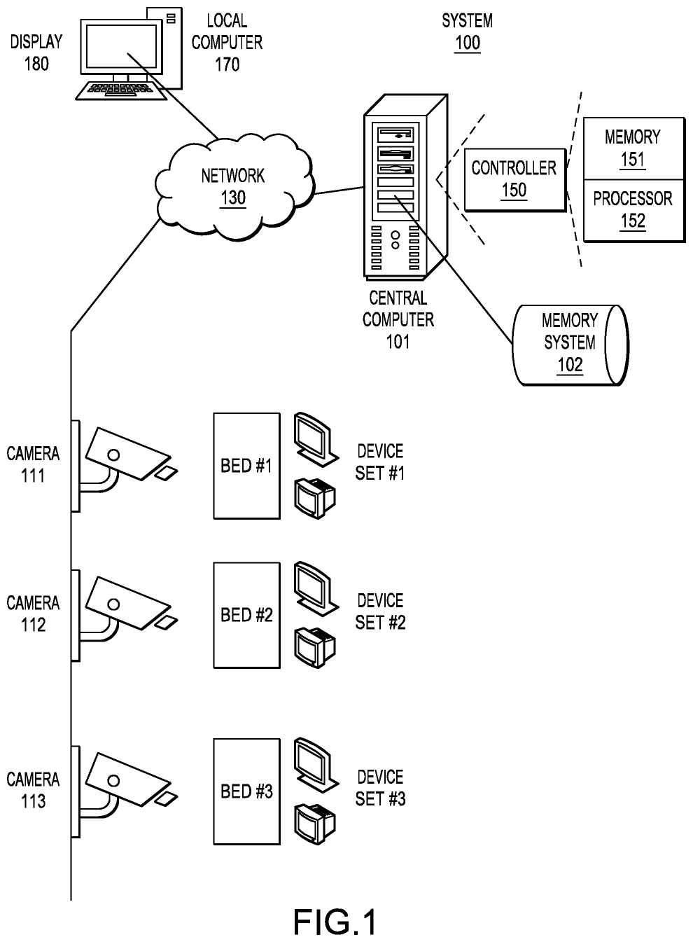

FIG. 1 illustrates a system 100 for enhancing data views for remote intensive care units, in accordance with a representative embodiment.

The system 100 in FIG. 1 is a system for enhancing data views for remote intensive care units and includes components that may be provided together or that may be distributed. The system 100 includes a central computer 101, a memory system 102, a local computer 170, a display 180, a network 130, a first camera 111, a second camera 112, and a third camera 113. The central computer includes a controller 150, and the controller 150 includes a memory 151 and a processor 152. The first camera 111 is provided at a bed #1 with a device set #1. The second camera 112 is provided at a bed #2 with a device set #2. The third camera 113 is provided at a bed #3 with a device set #3. The bed #1, the bed #2 and the bed #3 may all be provided at the same remote intensive care unit, or at different remote intensive care units. Similarly, the device sets may each include different types of medical devices.

The central computer 101 may be a server provided centrally, such as at a data center or at a central data processing facility for a business with multiple distributed locations. The central computer 101 may implement some or all aspects of methods described herein. For example, the controller 150 in the central computer 101 may control logical aspects of methods described herein which are attributed to the central computer 101. As one example, the central computer 101 may provide enhanced labelling services for distributed local computers (e.g., the local computer 170) at multiple different geographic locations. Additionally, the first camera 111, the second camera 112, and the third camera 113 may be provided for beds at different locations within a single intensive care unit or at different locations for multiple different intensive care units. While three cameras and a single instance of the local computer 170 are shown in FIG. 1, the teachings herein are applicable to embodiments with substantially more than three cameras and/or substantially more than a single instance of the local computer 170.

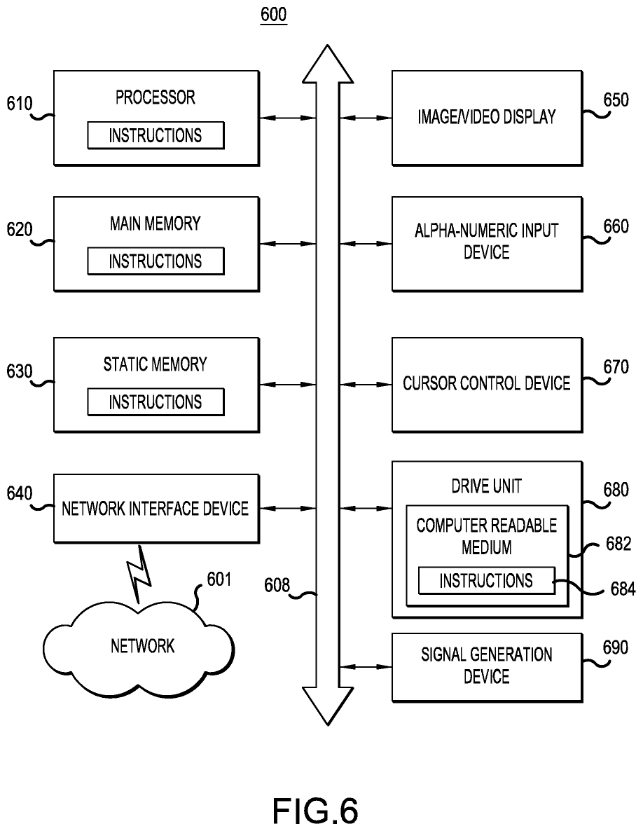

The controller 150 includes at least a memory 151 that stores instructions and a processor 152 that executes the instructions. A computer that can be used to implement the controller 150 is depicted in FIG. 6, though a controller 150 may include more or fewer elements than depicted in FIG. 1 or FIG. 6. In some embodiments, multiple different elements of the system 100 in FIG. 1 may include a controller such as the controller 150. For example, the central computer 101 and the local computer 170 may each include a controller with a combination of a memory that stores instructions and a processor that executes the instructions.

The local computer 170 is representative of the local computers at a remote intensive care unit, which provides remote intensive care for patients at the beds corresponding to the first camera 111, the second camera 112 and the third camera 113. The display 180 may be local to the local computer 170 or may be remotely connected to the local computer 170. The display 180 may be connected to the local computer 170 via a local wired interface such as an Ethernet cable or via a local wireless interface such as a Wi-Fi connection. The display 180 may be interfaced with other user input devices by which users can input instructions, including mouses, keyboards, thumbwheels and so on. The display 180 may be a monitor such as a computer monitor, a display on a mobile device, an augmented reality display, a television, an electronic whiteboard, or another screen configured to display electronic imagery. The display 180 may also include one or more input interface(s) that may connect other elements or components to the local computer 170, as well as an interactive touch screen configured to display prompts to users and collect touch input from users. The local computer 170 may receive video streams from the first camera 111, the second camera 112, and the third camera 113, and the display 180 may display the video streams with enhancements such as labels provided by the central computer 101.

Insofar as the display 180 may be used to display an enhanced video feed from one or more of the first camera 111, the second camera 112, and the third camera 113, the enhanced video feed may be allotted less than all of the screen of the display 180. For example, the video feed from a camera at a remote bed may be displayed on the display 180 along with other types of information including computer desktop information such as taskbars etc. Accordingly, the enhanced video feed may be provided relatively less space on the display 180 than on the user interface display of the medical device on which the data is being displayed at the remote bed. Labels for the video feed may be too small to recognize without the enhancements described herein, so the enhancements may be provided automatically, including when the enhancements are not particularly needed. The central computer 101 recognizes the type of the medical device, and extracts user interface elements such as labels, unit markers and their positions. The labels, unit markers and other types of information may be overlaid on the display 180 in the original layout of labels, making the labels, unit markers and other types of information clearly visible even if the portion of the display 180 allocated to the video stream from the bedside medical device is a relatively-small portion such as 30% of the screen of the display 180.

The controller 150 may perform some of the operations described herein directly and may implement other operations described herein indirectly. For example, the controller 150 may directly control operations such as logical operations performed by the processor 152 executing instructions from the memory 151 based on input received from electronic elements and/or users via the interfaces.

The central computer 101 may receive data generated by the first camera 111 at a first remote intensive care unit, and may also perform the same or similar processes for data received from the second camera 112 at a second remote intensive care unit, and may also perform the same or similar processes for data received from the third camera 113 at a third remote intensive care unit.

Additionally, each of the device sets #1, #2 and #3 may include multiple medical devices. For example, the device set #1 may include a first medical device of a type determined by the central computer 101. The device set #2 may include multiple devices such as a first medical device, a second medical device, and a third medical device, and the central computer 101 may determine the type(s) of each of the first medical device, the second medical device and the third medical device. The central computer 101 may perform processes for multiple different medical devices within a single device set, multiple different medical devices within different device sets at a single remote intensive care unit, and/or multiple different medical devices within different device sets at different remote intensive care units.

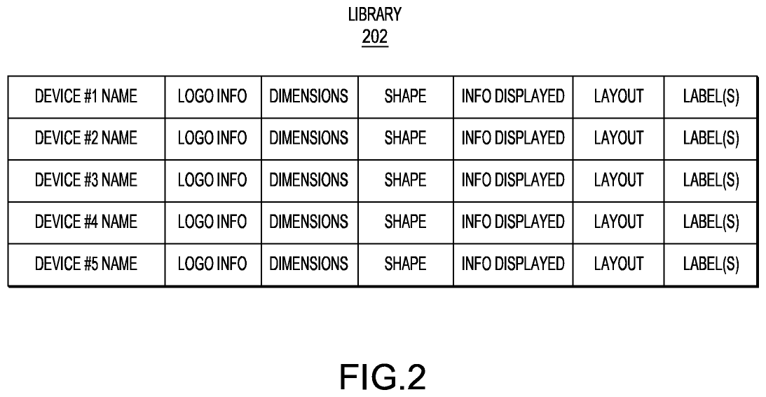

FIG. 2 illustrates a library for enhancing data views for remote intensive care units, in accordance with a representative embodiment.

The library 202 in FIG. 2 may be stored in the memory system 102 in FIG. 1. The library 202 may be a library of information for types of medical devices which may be captured in views of the cameras in FIG. 1. In the library 202 shown in FIG. 2, data for devices is arranged logically by device name, such as device name #1, device name #2, device name #3, device name #4, device name #4. In some embodiments, data for devices may be arranged by device type, device manufacturer, or in other groupings for devices.

The library 202 may be pre-built as a recognition library which stores and associates device names and layouts of layout information with visible characteristics that may be captured by the cameras in FIG. 1. The layouts may include user interface parameters for displays of the devices in the device sets. Additionally, the library 202 may include lists of medical devices used in particular ICUs and even for particular beds at ICUs, so that identification of the devices may be limited to a small set of potential medical devices.

In the library 202, each device is associated in a data set with logo information (logo info) for the device. The logo information may include graphical information corresponding to the shape and color of a logo for the device, and is used to visually identify the device by a logo captured by one of the first camera 111, the second camera 112 or the third camera 113. Logos may be used to identify devices, so that layouts of label information displayed by user interfaces of the devices may be determined when the devices are captured in the views of the first camera 111, the second camera 112 and the third camera 113.

In the library 202, each device is associated with dimensions for the device. The dimensions may include dimension characteristics for the device, such as overall height, width and depth (e.g., X, Y, Z coordinates relative to a predetermined origin). Also or alternatively, the dimensions may also include dimension characteristics for a user interface of the device, such as height and width of the user interface (e.g., X, Y coordinates relative to a predetermined origin). Dimensions may be used to identify devices, so that layouts of label information displayed by user interfaces of the devices can be determined when the devices are captured in the views of the first camera 111, the second camera 112 and the third camera 113.

In the library 202, each device is associated with shape for the device. The shape may include characteristics derived from dimensions for the device, such as whether the device comprises a rectangle or square or circle, as well as details such as ratios of the height and width of a user interface of the device. Shapes may be used to identify devices, so that layouts of label information displayed by user interfaces of the devices can be determined when the devices are captured in the views of the first camera 111, the second camera 112 and the third camera 113.

In the library 202, each device is associated with information displayed (info displayed) on a user interface, a layout of the label information displayed on the user interface, and labels for the information displayed on the user interface. For example, a layout of label information for an identified device may specify that SpO2 readings are displayed two inches below and two inches to the right of the upper left corner.

Although not shown in FIG. 2, the library 202 may also or alternatively store information corresponding to the user interface and/or layout of the device to be identified. For example, the number of and relative positioning of information elements corresponding to data items and labels may be used as the basis for identifying a type of the device. Additionally, when a single device has more than one user interface during monitoring, the number of and relative positioning of information elements of each of the multiple user interfaces may be used to recognize the type of the device. For example, one user interface may have two information elements and another user interface of the same device may have three information elements, and the proper labelling for each of the information elements may be determined and generated for each of the user interfaces.

As should be clear from the library 202 in FIG. 2 and the description thereof, a variety of ways to identify each device with characteristics stored in the library may be used. The ways to identify devices in the library 202 are not limited to those shown in FIG. 2, and not all of the ways shown in FIG. 2 are necessary to identify a device in each instance. For example, a device may be identified solely from a logo, or from a combination of logo and shape, or from a combination of logo and dimensions.

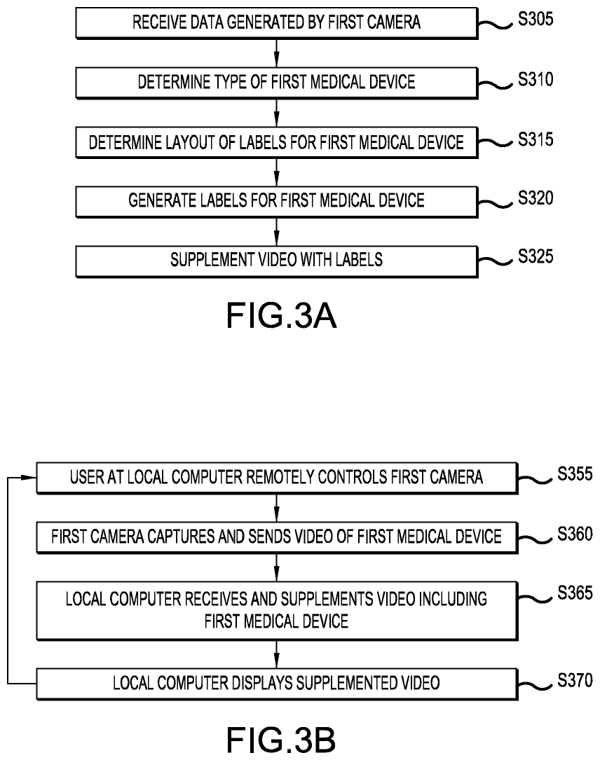

FIG. 3A illustrates a method for enhancing data views for remote intensive care units, in accordance with a representative embodiment.

The method of FIG. 3A may be performed by the central computer 101 and the memory system 102 in the system 100 in FIG. 1. At S305, the method of FIG. 3A includes receiving data generated by a first camera. The first camera from which data is received at S305 may be the first camera 111 in FIG. 1. The data may comprise image data such as video data. The received data may include imagery of the one or more devices in the device set #1 in FIG. 1. The data received at S305 may be a video stream of images/imagery captured by the first camera 111.

At S310, the method of FIG. 3A includes identifying a type of the first medical device from the data generated by the first camera 111. The type of the first medical device may include a brand and model, for example. The type of the first medical device may be identified from the library 202 in the memory system 102. For example, the type of the first medical device may be identified using image analysis, such as by an algorithm that searches for known shapes in or around a display of each device of the device set #1 that appears in a video feed from the first camera 111. The known shapes may correspond to logos, dimensions, shapes and any other characteristics of the devices of the device set that may be used to identify the type of the first medical device. Identification of the type of the first medical device may include a brand name, model, year of manufacture, manufacturer and other types of information that may identify the first medical device relative to other medical devices and/or types of medical devices. In some embodiments, a specific instance of a medical device such as a first medical device may be identified at S310. For example, barcodes or similar visualizable identifiers may be placed on medical devices so that the data generated by the first camera 111 may include the visualizable identifier. A visualizable identifier may specify a specific instance of a medical device, or more generally, the type of the specific instance of the medical device.

In some embodiments, an image analysis algorithm may be developed using one or more of the cameras shown in FIG. 1. For example, one of the cameras may be used to capture multiple images of the views of different medical devices from different angles and different zoom rates. In some embodiments, machine learning or deep learning methods may be used to recognize the medical devices in order to retrieve a device identification of each medical device. In some embodiments, image analysis algorithms may also detect boundaries of displays of the medical devices.

An image analysis algorithm may collect or generate the shape information from the data received from the first camera, compare the shape information to analogous shape information stored in a database of shapes corresponding to the devices in the device set of interest (#1), and identify the type of the device based on scoring how well the shape information from the data matches the shape information from the database. A threshold may be used to ensure that shape information with a top score from the database is identical or nearly identical to the corresponding shape information from the data received from the first camera.

As an example of an identification of a medical device in a device set, specifications and parameters of each type of medical device may be stored in the library 202. Additionally, information of the labels including content, position, and layout of label information may be stored in the library 202 for each medical device which may be identified by the image analysis at S310. In an example embodiment, a device identification may be a numerical identification such as 10001, and may be accompanied by resolution and position information for an item of data such as 100 mv, 10 pixels over and 10 pixels down (or up). A predetermined origin may be set as, for instance the upper left corner.

In some embodiments, one or more of the medical devices in a device set may be connected to the internet, and may identify themselves to the tele-ICU. In such embodiments, the device identification and data enhancement may be selectively implemented for medical devices which are not connected to the internet.

At S315, the method of FIG. 3A includes determining a layout of label information for the first medical device. The layout of label information may be retrieved from the library 202 based on the type of device identified at S310. The layout of label information may be a default arrangement of information shown on the first medical device, such as a location of pulse readings, a location of blood pressure readings, and a location of oxygen saturation readings on a display of the first medical device.

At S320, the method of FIG. 3A includes generating labels for the first medical device. The labels generated at S320 may be generated in a predetermined size and brightness that is larger and brighter than the labels for information generated by the first medical device itself. The labels may be specifically generated to assist viewers who may have trouble otherwise identifying what type of information they are seeing on a video feed that includes the display of the first medical device.

At S325, the method of FIG. 3A includes supplementing the video with labels. For example, the central computer 101 may superimpose the labels generated at S320 on a video feed from the first camera 111, and then provide the supplemented video to the local computer 170 over the network 130. Alternatively, the central computer 101 may simply generate the labels and locations for the labels generated at S320, and provide the labels and locations to the local computer 170 so that the local computer 170 may overlay the labels on the video as the video is displayed on the display 180.

As should be evident, the method of FIG. 3A may be performed in parallel by the central computer 101 for multiple cameras in one or more locations. The central computer 101 may be a server that dynamically identifies medical devices and supplements videos of the medical devices for users at a tele-ICU as described herein.

FIG. 3B illustrates a method for enhancing data views for remote intensive care units, in accordance with a representative embodiment.

At S355, the method of FIG. 3B includes a user at a local computer remotely controlling the first camera. The local computer may be the local computer 170 in FIG. 1. A user at a tele-ICU may use the local computer 170 to remotely interact with clinicians and patients at bed #1 in FIG. 1, such as via the camera 111. As an example, a user using the local computer 170 in FIG. 1 may view the display content of the devices in device sets at bedsides of patients in actual ICUs via a video stream. When a physician using the local computer 170 wants to observe a bedside medical device, the physician may use a pointing device such as mouse-controlled cursor to select the target bedside medical device part as region of interest in the video. The cameras described herein may include motors and may be controllably movable so that the cameras may be controlled to face devices in devices sets at patient bedsides. The cameras may also be programmable, such as by including memories that store instructions and processors that execute the instructions, may execute iterative loops to zoom in and out until features of the medical devices can be seen and recognized. Loops may include zooming in, zooming out or both, such as at predefined increasing steps. After the medical device is identified, the identification of the medical device may be used to retrieve and stream enhancement data such as labels from the central computer 101 to the local computer 170.

At S360, the method of FIG. 3B includes the first camera capturing and sending video of the first medical device. The first camera may capture and send the video to the central computer 101 in FIG. 1, such as via a service used by users of a tele-ICU that includes the local computer 170 in FIG. 1.

At S365, the method of FIG. 3B includes the local computer receiving and supplementing video including the first medical device. For example, the local computer 170 may receive labels generated by the central computer 101 as discussed above with reference to S325, and supplement video received directly or indirectly from the first camera 111 with the labels received from the central computer 101.

At S370, the method of FIG. 3B includes the local computer displaying the supplemented video. For example, the local computer 170 may generate and display supplemented video on the display 180 in FIG. 1. The supplemented video may include labels generated by the central computer 101 at locations of a layout of label information provided from the central computer 101 based on the data stored in the library 202 of the memory system 102.

Using the system 100 of FIG. 1 and the method of FIGS. 3A and 3B, user interface displays of devices in device sets may be streamed electronically from bedside to remote side. A user at the local computer 170 may review the enhanced streamed video on the display 180. The user at the local computer 170 may quickly and efficiently switch the video on demand from patient view to device view, and obtain enhanced video of the user interface displays of devices in device sets captured by the bedside cameras.

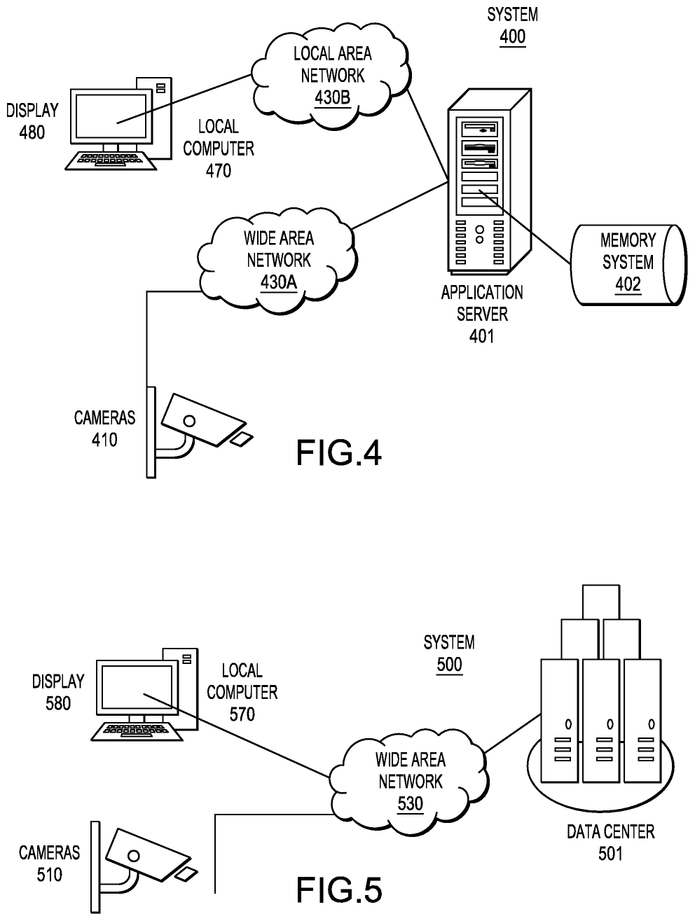

FIG. 4 illustrates a system for enhancing data views for remote intensive care units, in accordance with a representative embodiment.

In FIG. 4, a system 400 includes an application server 401 and a memory system 402, cameras 410, a local computer 470, a display 480, a wide area network 430A and a local area network 430B. The application server 401 and the memory system 402 may be provided at a central location with the local computer 470 and the display 480. For example, a tele-ICU facility may provide an application server 401 and memory system 402 for one or more teams of clinicians who provide remote medical services for patients at beds corresponding to the cameras 410. Such a central location may provide remote medical services for multiple different actual ICUs, and each of the multiple different actual ICUs may include multiple beds each corresponding to one or more of the cameras 410.

Each of the cameras 410 may correspond to a different device set with one or more devices. Each device in a device set in embodiments based on FIG. 4 may include a visual user interface that displays information which can be captured by one or more of the cameras 410. The assistance provided by the application server 401 and the memory system 402 may result in usable labels for the information displayed by the devices in the device sets.

FIG. 5 illustrates a system for enhancing data views for remote intensive care units, in accordance with a representative embodiment.

The system in FIG. 5 includes a data center 501, a local computer 570, a display 580 and cameras 510. The data center 501 may include multiple application servers and memory systems corresponding to the application server 401 and the memory system 402 in FIG. 4. In a cloud embodiment, enhancement services provided for tele-ICUs may be provided dynamically and on demand. Video feeds from the cameras 510 may be provided to the data center 501 so that the application servers may analyze imagery of the devices in the device sets captured by the cameras 510, and labels may be provided for the information displayed on the user interfaces of the devices in the device sets.

FIG. 6 illustrates a computer system, on which a method for enhancing data views for remote intensive care units is implemented, in accordance with another representative embodiment.

Referring to FIG. 6, the computer system 600 includes a set of software instructions that can be executed to cause the computer system 600 to perform any of the methods or computer-based functions disclosed herein. The computer system 600 may operate as a standalone device or may be connected, for example, using a network 601, to other computer systems or peripheral devices. In embodiments, a computer system 600 performs logical processing based on digital signals received via an analog-to-digital converter.

In a networked deployment, the computer system 600 operates in the capacity of a server or as a client user computer in a server-client user network environment, or as a peer computer system in a peer-to-peer (or distributed) network environment. The computer system 600 can also be implemented as or incorporated into various devices, such as a central computer that includes a controller, a local computer that includes a controller, a server that includes a controller, a camera, one or more devices of a device set, or any other machine capable of executing a set of software instructions (sequential or otherwise) that specify actions to be taken by that machine. The computer system 600 can be incorporated as or in a device that in turn is in an integrated system that includes additional devices. In an embodiment, the computer system 600 can be implemented using electronic devices that provide voice, video or data communication. Further, while the computer system 600 is illustrated in the singular, the term “system” shall also be taken to include any collection of systems or sub-systems that individually or jointly execute a set, or multiple sets, of software instructions to perform one or more computer functions.

As illustrated in FIG. 6, the computer system 600 includes a processor 610. The processor 610 may be considered a representative example of a processor of a controller and executes instructions to implement some or all aspects of methods and processes described herein. The processor 610 is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The processor 610 is an article of manufacture and/or a machine component. The processor 610 is configured to execute software instructions to perform functions as described in the various embodiments herein. The processor 610 may be a general-purpose processor or may be part of an application specific integrated circuit (ASIC). The processor 610 may also be a microprocessor, a microcomputer, a processor chip, a controller, a microcontroller, a digital signal processor (DSP), a state machine, or a programmable logic device. The processor 610 may also be a logical circuit, including a programmable gate array (PGA), such as a field programmable gate array (FPGA), or another type of circuit that includes discrete gate and/or transistor logic. The processor 610 may be a central processing unit (CPU), a graphics processing unit (GPU), or both. Additionally, any processor described herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems. The term computing device should also be interpreted to include a collection or network of computing devices each including a processor or processors. Programs have software instructions performed by one or multiple processors that may be within the same computing device or which may be distributed across multiple computing devices.

The computer system 600 further includes a main memory 620 and a static memory 630, where memories in the computer system 600 communicate with each other and the processor 610 via a bus 608. Either or both of the main memory 620 and the static memory 630 may be considered representative examples of a memory of a controller, and store instructions used to implement some or all aspects of methods and processes described herein. Memories described herein are tangible storage mediums for storing data and executable software instructions and are non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The main memory 620 and the static memory 630 are articles of manufacture and/or machine components. The main memory 620 and the static memory 630 are computer-readable mediums from which data and executable software instructions can be read by a computer (e.g., the processor 610). Each of the main memory 620 and the static memory 630 may be implemented as one or more of random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, blu-ray disk, or any other form of storage medium known in the art. The memories may be volatile or non-volatile, secure and/or encrypted, unsecure and/or unencrypted.

“Memory” is an example of a computer-readable storage medium. Computer memory is any memory which is directly accessible to a processor. Examples of computer memory include, but are not limited to RAM memory, registers, and register files. References to “computer memory” or “memory” should be interpreted as possibly being multiple memories. The memory may for instance be multiple memories within the same computer system. The memory may also be multiple memories distributed amongst multiple computer systems or computing devices.

As shown, the computer system 600 further includes a video display unit 650, such as a liquid crystal display (LCD), an organic light emitting diode (OLED), a flat panel display, a solid-state display, or a cathode ray tube (CRT), for example. Additionally, the computer system 600 includes an input device 660, such as a keyboard/virtual keyboard or touch-sensitive input screen or speech input with speech recognition, and a cursor control device 670, such as a mouse or touch-sensitive input screen or pad. The computer system 600 also optionally includes a disk drive unit 680, a signal generation device 690, such as a speaker or remote control, and/or a network interface device 640.

In an embodiment, as depicted in FIG. 6, the disk drive unit 680 includes a computer-readable medium 682 in which one or more sets of software instructions 684 (software) are embedded. The sets of software instructions 684 are read from the computer-readable medium 682 to be executed by the processor 610. Further, the software instructions 684, when executed by the processor 610, perform one or more steps of the methods and processes as described herein. In an embodiment, the software instructions 684 reside all or in part within the main memory 620, the static memory 630 and/or the processor 610 during execution by the computer system 600. Further, the computer-readable medium 682 may include software instructions 684 or receive and execute software instructions 684 responsive to a propagated signal, so that a device connected to a network 601 communicates voice, video or data over the network 601. The software instructions 684 may be transmitted or received over the network 601 via the network interface device 640.

In an embodiment, dedicated hardware implementations, such as application-specific integrated circuits (ASICs), field programmable gate arrays (FPGAs), programmable logic arrays and other hardware components, are constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

Accordingly, enhancing data views for remote intensive care units provides for enhanced data view for tele-ICU systems. The enhancement provided by a centralized computer such as a server may result in improved outcomes for medical treatments, and is based on identifying types of devices on which data is shown in a remote location so that the nature of the data may be understood by personnel at the remote tele-ICU systems. Using bedside cameras to capture and recognize medical devices that remote physicians want to observe, the captured video of the user interface displays of the medical devices may be streamed and enhanced with detailed labels and unit markers overlaid on the video. The enhanced labels may provide an improved observation ability for physicians, and allow improved medical efficacy and user experiences.

Although enhancing data views for remote intensive care units has been described with reference to several exemplary embodiments, it is understood that the words that have been used are words of description and illustration, rather than words of limitation. Changes may be made within the purview of the appended claims, as presently stated and as amended, without departing from the scope and spirit of enhancing data views for remote intensive care units in its aspects. Although enhancing data views for remote intensive care units has been described with reference to particular means, materials and embodiments, enhancing data views for remote intensive care units is not intended to be limited to the particulars disclosed; rather enhancing data views for remote intensive care units extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72 (b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

We claim:

1. A system for enhancing data views for remote intensive care units, comprising:
a central computer comprising a memory that stores instructions and a processor that executes the instructions, wherein, when executed by the processor, the instructions cause the central computer to:
receive, from a first remote intensive care unit, data generated by a first camera at the first remote intensive care unit;
determine, from the data generated by the first camera at the first remote intensive care unit, a type of a first medical device within a view of the first camera at the first remote intensive care unit;
determine, based on the type of the first medical device, a layout of labels for information displayed by the first medical device;
generate labels for information displayed by the first medical device; and
supplement video from the first camera with the labels for the information displayed by the first medical device.

2. The system of claim 1, further comprising:
a memory system that stores a library of information for types of medical devices including the first medical device.

3. The system of claim 1, further comprising:
a memory system that stores a layout of label information of user interfaces of medical devices including the first medical device;
wherein, when executed by the processor, the instructions cause the central computer further to:
determine, based on the type of the first medical device, the layout of label information of a user interface on the first medical device.

4. The system of claim 1, wherein the first medical device is not connected to the internet and the first camera is connected to the internet and communicates over the internet with the central computer.

5. The system of claim 1, wherein, when executed by the processor, the instructions cause the central computer further to:
provide, to a local computer, the video from the first camera supplemented with the labels for information displayed by the first medical device.

6. The system of claim 5, wherein, when executed by the processor, the instructions cause the central computer further to:
provide the labels to the local computer to overlay on the video from the first camera to label information displayed by the first medical device and captured by the first camera.

7. The system of claim 1, wherein, when executed by the processor, the instructions cause the central computer further to:
receive, from a second remote intensive care unit, data generated by a second camera at the second remote intensive care unit;
determine, from the data generated by the second camera at the second remote intensive care unit, a type of a second medical device within a view of the second camera at the second remote intensive care unit;
determine, based on the type of the second medical device, a layout of labels for information displayed by the second medical device;
generate labels for information displayed by the second medical device; and
supplement video from the second camera with the labels for information displayed by the second medical device.

8. The system of claim 1, wherein, when executed by the processor, the instructions cause the central computer further to:
determine, from the data generated by the first camera at the first remote intensive care unit, a type of a second medical device within a view of the first camera at the first remote intensive care unit;
determine, based on the type of the second medical device, a layout of labels for information displayed by the second medical device;
generate labels for information displayed by the second medical device; and
supplement the video from the first camera with the labels for information displayed by the second medical device.

9. A method for enhancing data views for remote intensive care units, the method comprising:
receiving, by a central computer comprising a memory that stores instructions and a processor that executes the instructions, from a first remote intensive care unit, data generated by a first camera at the first remote intensive care unit;
determining, from the data generated by the first camera at the first remote intensive care unit, a type of a first medical device within a view of the first camera at the first remote intensive care unit;
determining, based on the type of the first medical device, a layout of labels for information displayed by the first medical device;
generating labels for information displayed by the first medical device; and
supplementing video from the first camera with the labels for the information displayed by the first medical device.

10. The method of claim 9, further comprising:
storing a library of information for types of medical devices including the first medical device.

11. The method of claim 9, further comprising:
storing a layout of label information of user interfaces of medical devices including the first medical device; and
determining, based on the type of the first medical device, the layout of label information of a user interface on the first medical device.

12. The method of claim 9, wherein the first medical device is not connected to the internet and the first camera is connected to the internet and communicates over the internet with the central computer.

13. The method of claim 9, further comprising:
providing, to a local computer, the video from the first camera supplemented with the labels for information displayed by the first medical device.

14. The method of claim 9, further comprising:
providing the labels to a local computer to overlay on the video from the first camera to label information displayed by the first medical device and captured by the first camera.

15. The method of claim 9, further comprising:
receiving, from a second remote intensive care unit, data generated by a second camera at the second remote intensive care unit;
determining, from the data generated by the second camera at the second remote intensive care unit, a type of a second medical device within a view of the second camera at the second remote intensive care unit;
determining, based on the type of the second medical device, a layout of labels for information displayed by the second medical device;
generating labels for information displayed by the second medical device; and
supplementing video from the second camera with the labels for information displayed by the second medical device.

16. The method of claim 9, further comprising:
determining, from the data generated by the first camera at the first remote intensive care unit, a type of a second medical device within a view of the first camera at the first remote intensive care unit;
determining, based on the type of the second medical device, a layout of labels for information displayed by the second medical device;
generating labels for information displayed by the second medical device; and
supplementing the video from the first camera with the labels for information displayed by the second medical device.

17. A tangible non-transitory computer readable storage medium that stores a computer program, wherein the computer program, when executed by a processor, causes a central computer to:
receive, from a first remote intensive care unit, data generated by a first camera at the first remote intensive care unit;
determine, from the data generated by the first camera at the first remote intensive care unit, a type of a first medical device within a view of the first camera at the first remote intensive care unit;
determine, based on the type of the first medical device, a layout of labels for information displayed by the first medical device;
generate labels for information displayed by the first medical device; and
supplement video from the first camera with the labels for the information displayed by the first medical device.

18. The tangible non-transitory computer readable storage medium of claim 17, wherein the computer program, when executed by a processor, causes the central computer further to:
receive, from a second remote intensive care unit, data generated by a second camera at the second remote intensive care unit;
determine, from the data generated by the second camera at the second remote intensive care unit, a type of a second medical device within a view of the second camera at the second remote intensive care unit;
determine, based on the type of the second medical device, a layout of labels for information displayed by the second medical device;
generate labels for information displayed by the second medical device; and
supplement video from the second camera with the labels for information displayed by the second medical device.

19. The tangible non-transitory computer readable storage medium of claim 17, wherein the computer program, when executed by a processor, causes the central computer further to:
determine, from the data generated by the first camera at the first remote intensive care unit, a type of a second medical device within a view of the first camera at the first remote intensive care unit;
determine, based on the type of the second medical device, a layout of labels for information displayed by the second medical device;
generate labels for information displayed by the second medical device; and
supplement the video from the first camera with the labels for information displayed by the second medical device.
